(19) United States
(12) Patent Application Publication
(10) Pub. No.: US 2013/0069365 A1
(43) Pub. Date: Mar. 21, 2013

(54) Tapered Bore Connector

(71) Applicant: James L. Pokorney, Northfield, MN (US)

(72) Inventor: James L. Pokorney, Northfield, MN (US)

(21) Appl. No.: 13/603,919

(22) Filed: Sep. 5, 2012

Related U.S. Application Data
(60) Provisional application No. 61/573,466, filed on Sep. 6, 2011.

(51) Int. Cl. F16L 37/244

(52) U.S. Cl. CPC F16L 37/2445
USPC 285/333

(57) ABSTRACT

The invention is a tapered bore connector comprising a body having a conical bore with a central axis that forms an opening in the body. At least one compound cantilevered flange is attached to the body and extends a predetermined distance from the body in an outward radial direction relative to the conical bore axis and also extends a predetermined distance from the body in a circumferential direction relative to the conical bore axis.

In operation, when the tapered bore connector is fully engaged with a compatible tapered shaft connector having an internally threaded locking collar, the compound cantilevered flange is deflected thereby generating an additional restraining force that dissuades premature disconnection.

This application claims priority from provisional patent application U.S. Ser. No. 61/573,466 filed Sep. 6, 2011.

### Background

1. Field of Invention

This invention relates to an improved tapered bore connector.

2. Definitions

Circumferential Direction—A direction following a circumference or perimeter about a center or axis.

Radial Direction—A direction radiating outward from a center or axis.

Helix Angle—The angle of a thread or flange traversing in a helix around an axis relative to a plane perpendicular to the axis. If a flange is horizontal, the helix angle is zero.

Double Threaded—Two parallel threads on the same locking collar or luer lock sleeve one of which is 180° ahead of the other.

### BACKGROUND

A tapered bore connector is a connector designed to mate with a compatible tapered shaft connector. Tapered bore connectors are used in many industrial applications including machine tools, medical devices, and industrial piping. A particular tapered bore connector is used in fluid and gas applications and is known as a luer connector. A luer connector is designed for small-scale fluid fittings to create leak-free connections between a female taper fitting and its mating male part and is widely used on medical and laboratory instruments. Named after the 19th century German medical instrument maker Hermann Wülfing Luer, the luer connection originated as a 6% taper fitting for glass bottle stoppers.

The modern luer fitting includes a third element, an internal double threaded sleeve or collar with a helix angle of about 25 to 30 degrees. This collar is positioned around the male luer. It is intended to engage with two mating lugs or flanges attached to the body of the female luer connector. The lugs or flanges traverse in a spiral fashion along the threads until the tapered bore and shaft fully mate. Further tightening causes the flanges and threads to slightly deflect causing an interference fit. The locking collar concept, sometimes called a luer lock, was invented by Fairleigh Dickinson in 1929 and is described in his patent U.S. Pat. No. 1,793,068. The Dickinson invention is arguably one of the building blocks that enabled the modern medical device industry to grow and flourish. Using his luer design, medical devices such as needles, catheters, syringes, IV bags, stopcocks, fluid pumps, pressure transducers, and other devices are interchangeably connected to each other following a simple rule accepted by industry. At the distal end of a medical device a male luer device is attached. At the proximal end of a device a female luer device is attached. Using the Dickinson luer design and following this male/distal and female/proximal connector convention, the customer is assured that devices manufactured by different suppliers can be connected together in a safe, predictable, and convenient way.

In 1991, the fundamental features of the Dickinson luer design were so engrained in the industry that the basic luer design was agreed upon by industry and hospital representatives and then codified and published as an international standard by the International Organization for Standardization (ISO). The standard is entitled ISO 594-2:1991, Conical fittings with a 6% (Luer) Taper for syringes, needles and certain other medical equipment.

Although intended to make a more secure fitting, the industry standard luer design has a major deficiency. Dickinson assumed a metal connector. This seems obvious based on Dickinson's selection of a steeply pitched internal double threads having a helix angle of about twenty five to thirty degrees. Because the Dickinson luer was fabricated of metal, the steeply threaded connector was most likely needed to release a tight fitting metal taper to metal taper connections at the end of a procedure. If only a strong connection was Dickinson's major design goal, a finer pitch thread would have been obvious to those skilled in the art like Dickinson. So it might be assumed that Dickinson compromised his design to help support both connection and disconnection of metal luer fittings. In 1929 Dickenson most likely did not anticipate the broad use of plastic disposable luer connectors and therefore he did not focus his design on maintaining a tight interference fit over extended time periods using less stable plastic materials.

It is now well known in the art of luer design that plastic connectors exhibit creep over time and that this stress relaxation diminishes the taper to taper hoop stresses—resulting in a loose connection. When this interference stress is diminished, the interference fit is relieved and the female luer is free to rotate relative to the male luer threads. Free rotation combined with the steep thread pitch allows for easy disassembly causing fluid leakage or air inflow. Almost all connectors are now made of plastic. In particular, many connectors are composed of polycarbonate, PVC, Acrylic, ABS or other plastic materials. Compared to metal connectors, all of these materials exhibit significant creep after being stressed for a long period of time.

The ISO luer standard specifically calls out design standards for a cantilevered lug or flange extending in the radial direction but does not specify or anticipate a cantilevered flange or lug extending in the circumferential direction.

### OBJECTS AND ADVANTAGES

The new tapered bore connector maintains a restraining friction fit between the internal threads of a tapered shaft connector and flange extending from a tapered bore connector even if substantial plastic creep occurs. The new invention meets the following design objectives:

1. Provides an improved interference fit between a tapered bore connector flange and the threaded collar male of a tapered shaft connector.

2. Complies with design parameters specified in ISO standard 594-2.

3. Does not significantly increase the cost of the tapered bore connector. In particular, the improved design does not require an additional part or any modification to the mating male connector.

4. Another object of this new invention is to prevent the newly invented compound cantilevered flange from excessive bending during operation.

The above mentioned objects and advantages of this invention will become apparent from the following description taken in connection with the accompanying drawings, wherein is set forth by way of illustration and example, preferred embodiments of this invention.

### DESCRIPTION OF DRAWING FIGURES

In the drawings, closely related figures have the same number but different alphabetic prefixes.

FIG. 1A shows a perspective view of a prior art tapered bore connector.

FIG. 1B shows a perspective view of a prior art tapered shaft connector

FIG. 2 shows a perspective view of a prior art tapered bore connector.

FIG. 3A-3B shows a schematic representation of prior art connector engagement

FIG. 4 shows a perspective view of a preferred embodiment of the invention

FIG. 5A-5D shows a schematic representation of a preferred embodiment connector engagement

FIG. 6 shows a perspective view of a preferred embodiment of the invention

FIG. 7 shows a perspective view of a preferred embodiment of the invention.

FIG. 8A-8C shows a schematic representation of a preferred embodiment connector engagement

FIG. 9 shows a perspective view of a preferred embodiment

FIG. 10 shows a perspective view of a preferred embodiment

### GENERAL SUMMARY OF INVENTION

The invention is a tapered bore connector comprising a body having a conical bore with a central axis that forms an opening in the body. At least one compound cantilevered flange is attached to the body and extends a predetermined distance from the body in an outward radial direction relative to the conical bore axis and also extends a predetermined distance from the body in a circumferential direction relative to the conical bore axis.

In operation, when the tapered bore connector is fully engaged with a compatible tapered shaft connector having an internally threaded locking collar, the compound cantilevered flange is deflected thereby generating an additional restraining force that dissuades premature disconnection.

To limit deflection of the compound cantilevered flange, in one embodiment a traditional radial cantilevered flange is also attached to the connector body. The helix angle of the bottom edge of the compound cantilevered flange is less than the helix angle of the radial cantilevered flange to allow for a more controlled deflection of the compound cantilevered flange.

### PRIOR ART

In prior art, the flange (or flanges) is cantilevered in the radially direction to allow the flange to engage with the internal threads of the collar of a mating male connector. The internal dimension of the mating locking collar limits the length of the radial cantilever. When fully engaged bore to taper, the flange is in close contact with the threads. When overtightened, the flange and threads tend to slightly bend or deform. As plastic deformation or creep occurs in the stressed plastic components, the interference fit is relaxed but the deformed flange acts like a spring to urge the two components together. Unfortunately, in many instances due to the relatively short radial cantilever arm, the amount of stored energy in the flange deformation is insufficient relative to the plastic deformation or creep. Over time, the interference fit is lost and the connectors can separate.

In FIG. 1A is a drawing of a Traditional Tapered Bore Connector 2. This prior art design has two Radial Cantilevered Flanges 5 attached to a Body 10. An Opening 15, not shown, is formed on the surface of Body 10. The Opening 10 in this prior art example is a Conical Bore 20, also not shown, transitioning to a Hole 22. In use, the Flanges 5 and Conical Bore 20 engage with a mating Male Conical Connector 24 similar to the one shown in FIG. 1B. The Male Conical Connector 24 has a Tapered Shaft 26 surrounded by a Locking Collar 27 with Internal Threads 28.

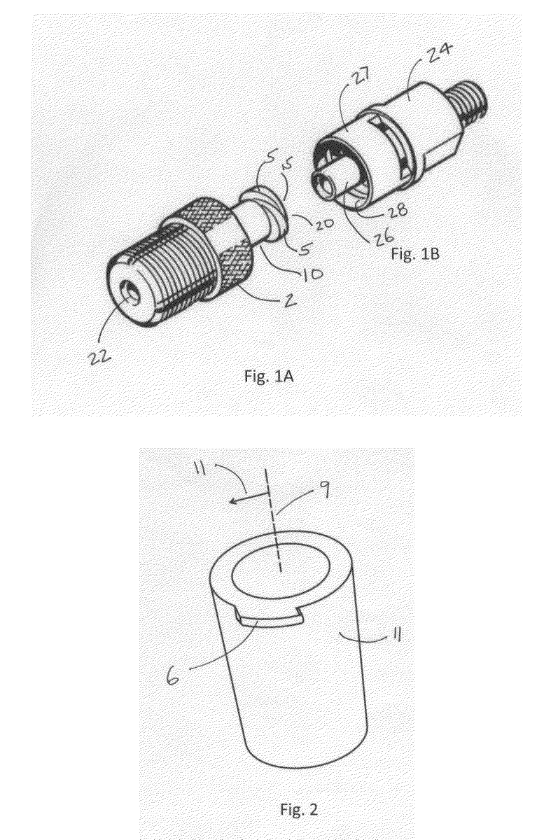

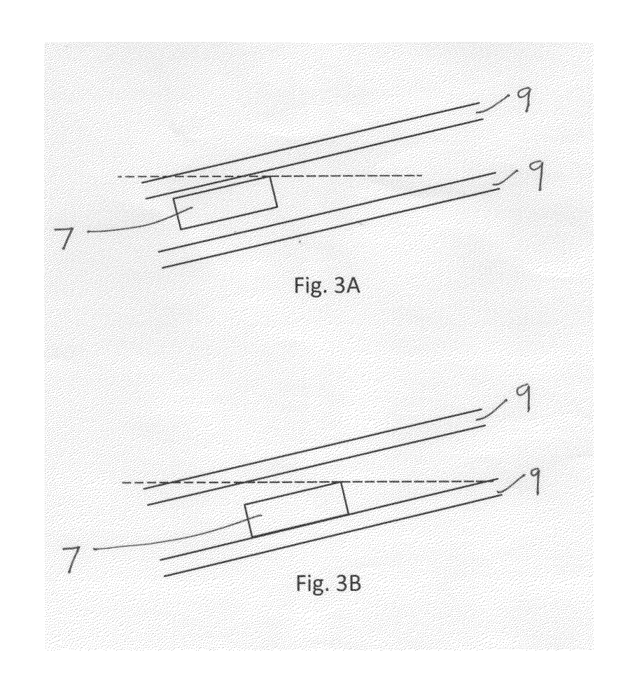

In FIG. 2 is a representation of a prior art of a Simple Radial Cantilevered Flange 6 attached to a Standard Body 11. The Simple Radial Cantilevered Flange 6 is only cantilevered in the radial direction relative to an Axis Reference Line 9. An Outward Radial Reference Arrow 11 depicts the general outward radial direction away from the Axial Reference Line 9. Only one flange is shown to simplify for the reader the basic design, although in many, perhaps most applications, two flanges are employed and are spaced about 180 degrees apart relative to the Axis Reference Line 9.

In FIG. 3A and 3B are schematic representations of the engagement of a prior art traditional radial cantilevered flange with the internal threads of a mating connector. In FIG. 3A, Schematic Radial Cantilevered Flange 7 is shown freely sliding within Schematic Internal Threads 9. In FIG. 3B the connectors are depicted in a fully engaged position. Flange 7 is in full contact with Schematic Thread 9. The dotted lines indicate edges of the tapered connector not in contact with the threads.

A prior art design is published as an international standard by the International Organization for Standardization (ISO) and is entitled ISO 594-2 : 1991, Conical fittings with a 6% (Luer) Taper for syringes, needles and certain other medical equipment.

### DETAILED DESCRIPTION

The new connector is comprised of a body element, a compound cantilever element, and in some embodiments a radial cantilever element. The connector can be made of plastic or metal. In preferred embodiments, all elements of the connector would be fabricated as a monolithic device using an injection molding process, a sintering process, or perhaps a discrete 3-D deposition process or some other assembly method known to those in the art of connector fabrication.

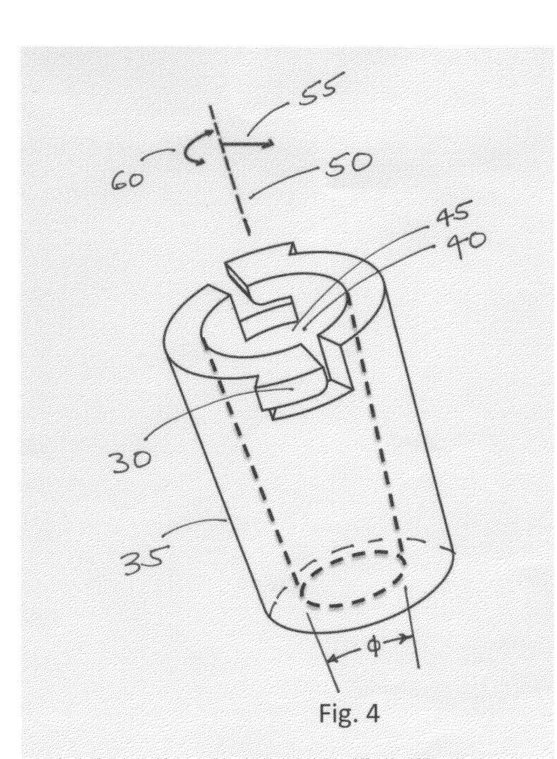

In preferred embodiments, the body element is generally cylindrical in shape, but in other embodiments it could be almost any shape. Preferred embodiments of a Body 35 are shown in FIG. 4. An Opening 45 is formed on the surface of Body 35. The Opening 45 is a hole or in this preferred embodiment a Conical Bore 40. The Conical Bore 40 establishes an Axial Reference Line 50. The Conical Bore 40 as shown by dotted lines is tapered along its length. In a preferred embodiment, the Conical Bore 40 has an Included Angle Φ. In a preferred embodiment Included Angle Φ is between five degrees and seven degrees and in a preferred embodiment the tapered bore transitions to a pathway that continues to make a continuous passageway through the body. Opening 45 can be of almost any diameter compatible with a mating connector. In a preferred embodiment the diameter is between 3 and 6 mm.

On the body near the conical bore opening is located one or more compound cantilevered flanges. A compound cantilevered flange can be generally described as a projecting beam or member that is supported at only one end and extends or cantilevers in a first direction and then further extends or cantilevers in a second direction. A compound cantilevered flange can be further described as a projecting beam or member having support at only one end and extending a predetermined distance in a circumferential direction and in a predetermined distance in an outward radial direction. The thickness and width of the flange, along with is length determines the stiffness of the flange according to general engineering formulas.

In preferred embodiments, the compound cantilevered flange element when combined with the body element is designed to engage with the internal threaded collar of a predetermined compatible tapered shaft connector.

A preferred embodiments of this compound flange invention is shown in FIG. 4. A Compound Cantilever Flange 30 is attached to Body 35. The Compound Cantilevered Flange 30 extends from Body 35 in an outward radial direction relative to Axial Reference Line 50. An Outward Radial Reference Arrow 55 is established to depict this general outward radial direction away from the Axial Reference Line 50. The Compound Cantilevered Flange 30 also extends from the Body 35 in a circumferential direction relative to the Axial Reference Line 50. A Circumferential Reference Arrow 60 is established to depict this general circumferential direction, traversing in either a clockwise or counterclockwise manner around the Axis Reference Line 50.

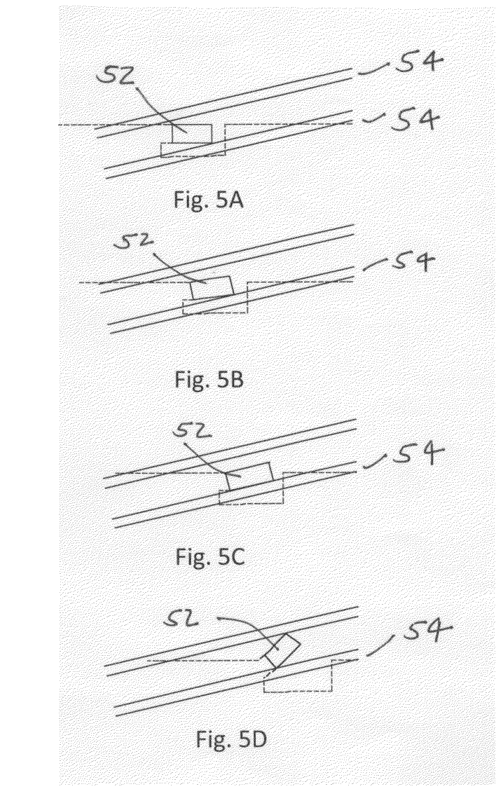

In FIG. 5A-5D are schematic representations showing the engagement of this newly invented compound cantilevered flange with the internal threads of a traditional mating connector. Again, the dotted lines indicate edges of the tapered conical connector that are not in contact with the threads.

In FIG. 5A Schematic Compound Cantilevered Flange 52 is shown freely sliding within Schematic Internal Threads 54. There is no restraining force generated by the Flange 52.

In FIG. 5B, the connectors are partially engaged. The Schematic Compound Cantilevered Flange 52 is shown partially deflected and almost flush to Schematic Internal Threads 54. There is some restraining force generated by the interference fit between Flange 52 and Thread 54.

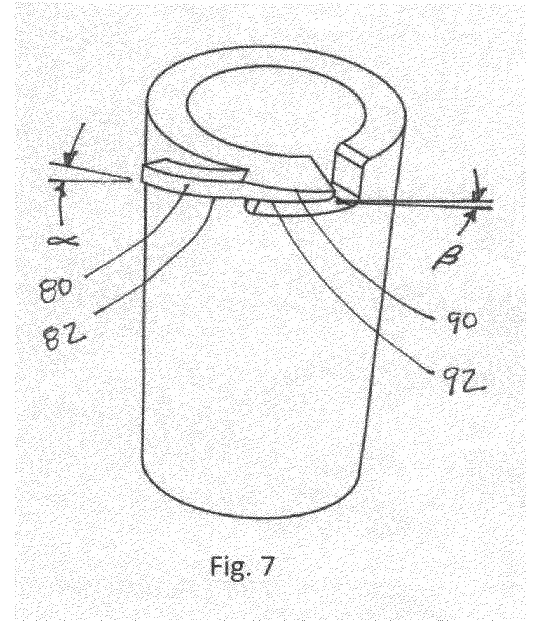

In FIG. 5C, the Schematic Compound Cantilevered Flange 52 is deflected such that it is laying flat with reference to the Schematic Internal Threads 54. Assuming Hooke's law of springs, the restraining force is now larger than that generated in the FIG. 7B schematic because the flange has been further deflected.

In FIG. 5D, the Schematic Compound Cantilevered Flange 52 is further deflected such that it no longer Is laying flat with reference to the Schematic Internal Threads 54. In this position, the flange may be over deflected, perhaps to the point of failure, depending on the specific flange dimensions and material properties selected.

To summarize, in operation the internal threads of a compatible tapered shaft connector deflect one or more compound cantilevered flanges when the tapered connectors are fully engaged. The deflected flange or flanges generate a restraining force that dissuades premature rotation leading to premature disconnection. In this embodiment, excessive deflection may occur.

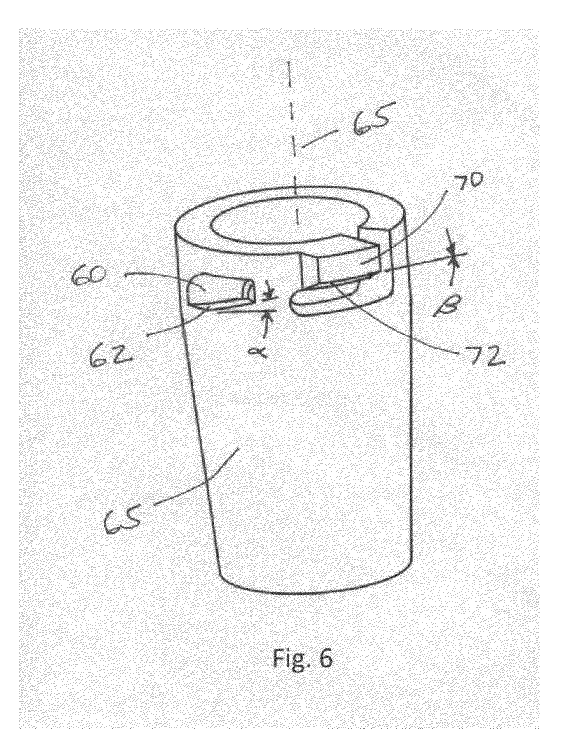

In another preferred embodiments, the tapered bore connector also comprises at least one radial cantilevered flange attached to the body. Embodiments of a radial cantilevered flange used with a compound cantilevered flange are shown in FIGS. 6,7, 9, and 10.

In FIG. 6 a Radial Cantilevered Flange 60 is cantilevered only in the radial direction relative to an Axis Reference Line 65 and is located on Body 65 near a Compound Cantilevered Flange 70. The Radial Cantilevered Flange 60 has a Radial Flange Bottom Edge 62 that generates a Helix angle α. In a preferred embodiment, the Helix angle α is between twenty five and thirty degrees, similar to that postulated by Dickinson in the original luer design. The Compound Cantilevered Flange 70 has a Compound Flange Bottom Edge 72 that generates a Helix angle β. In a preferred embodiments, the Helix Angle β is between about zero degrees and five degrees. In all embodiments, the Helix angle α is equal or greater than Helix angle β so that when the tapered bore connector is engaging with a compatible tapered shaft connector having an internally threaded locking collar, Compound Cantilevered Flange 70 is engaged first and deflects a predetermined deflection distance and then radial cantilevered flange is engaged second and deflects a second limited deflection distance to limit any further deflection of the compound cantilevered flange. In a preferred embodiment, the Helix Angle α of the Radial Flange Bottom Edge 62 is about equal to the helix angle of the threads located in a mating internal locking collar.

In FIG. 7 is another embodiment of the invention. A Radial Cantilevered Flange 80 is located closely adjacent to a Compound Cantilevered Flange 90 to form one monolithic flange. The Radial Cantilevered Flange 80 has a Radial Flange Bottom Edge 82 that generates a Helix angle α. The Compound Cantilevered Flange 90 has a Compound Flange Bottom Edge 92 that has a Helix angle β. Helix angle α is equal or greater than Helix angle β so that Compound Cantilevered Flange 90 is first contacted by the internal threads of a mating locking collar to cause a sufficient deflection distance of the Compound Cantilevered Flange 80 before the Radial Cantilevered Flange 80 is second contacted by the internal threads of the mating locking collar to limit any further deflection of the compound cantilevered flange. This embodiment prevents excessive deflection of the cantilevered flange as schematically shown in FIG. 5d, thereby preventing excessive plastic deformation or stress cracking of the compound cantilevered flange.

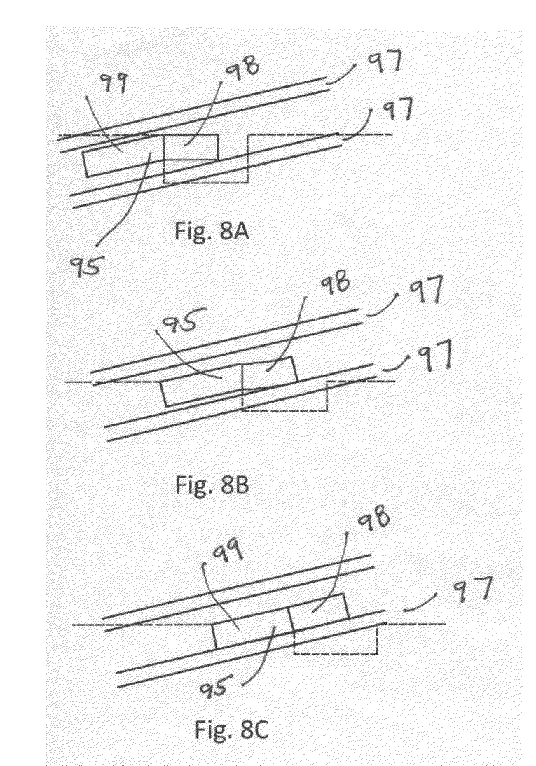

In FIG. 8A-8C are schematic representations showing the engagement of a compound cantilever flange closing adjacent the radial cantilever flange with the internal threads of a traditional mating connector. Again, the dotted lines indicate edges of the tapered conical connector that are not in contact with the threads.

In FIG. 8A, Schematic Compound/Radial Cantilevered Flange 95 is shown freely sliding within Schematic Internal Threads 97. It comprises Schematic Compound Cantilevered Flange Section 98 and a Schematic Radial Cantilevered Flange Section 99. There is no restraining force generated by the Flange 95.

In FIG. 8B, the connectors are partially engaged. The Schematic Compound Cantilevered Flange Section 98 is shown partially deflected and almost flush to Schematic Internal Threads 97. There is some restraining force generated by the interference fit between Flange 95 and Thread 97.

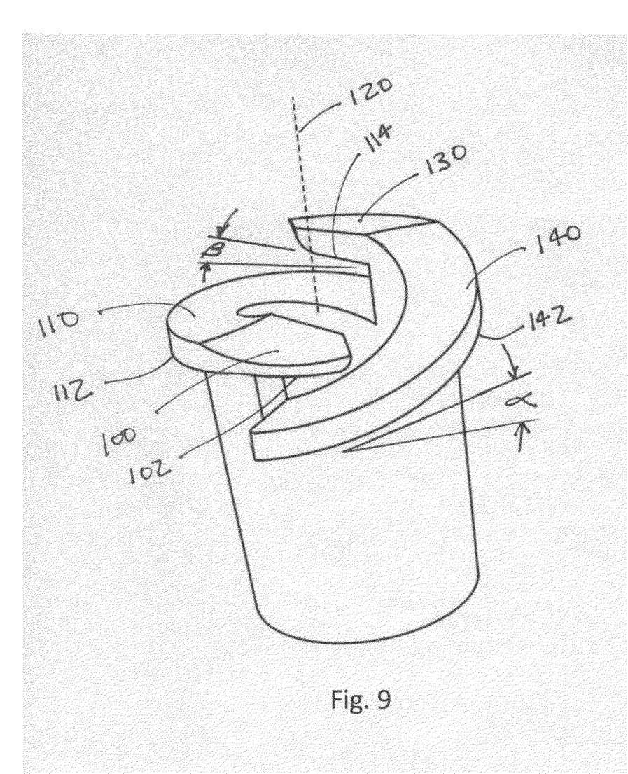

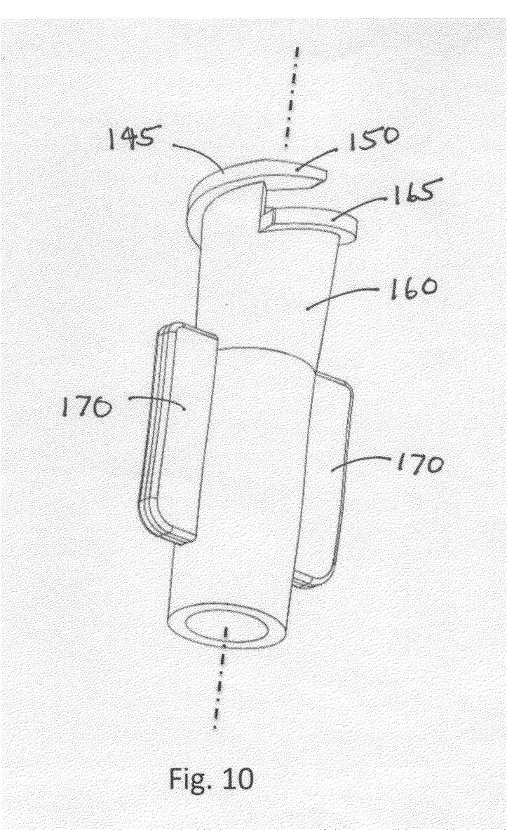

In FIG. 8C, the Schematic Compound Cantilevered Flange 95 is deflected such that it is laying flat with reference to the Schematic Internal Threads 97. Assuming Hooke's law of springs, the restraining force is now larger than that generated in the FIG. 9B schematic because the flange has been further deflected. Further deflection of the Schematic Compound Cantilevered Section 98 of the Schematic Compound Cantilevered Flange 95 is limited due to the abutment of the Schematic Radial Cantilevered Flange Section 99 with the Schematic Internal Threads 97.

In FIG. 9 is shown a preferred embodiment of the invention employing a Compound Cantilevered Flange 100 closely adjacent to Radial Cantilevered Flange 110 and spaced about 180 degrees relative an Ax is Reference Line 120 is a Compound Cantilevered Flange 130 closely adjacent to Radial Cantilevered Flange 140. The Radial Cantilevered Flanges 110 and 140 have a Radial Flange Bottom Edge 112 and 142 that generate a Helix angle α. The Compound Cantilevered Flange 100 and 130 have a Compound Flange Bottom Edge 102 and 114 that generate a Helix angle β. Helix angle α is equal or greater than Helix angle β so that Compound Cantilevered Flanges 110 and 130 are first contacted by the internal threads of a mating locking collar to cause a sufficient deflection distance of the Compound Cantilevered Flanges 110 and 130 before the Radial Cantilevered Flanges 110 and 140 are second contacted by the internal threads of the mating locking collar to limit any further deflection of the Compound Cantilevered Flanges 110 and 130.

FIG. 10 shows a preferred embodiment of the tapered conical connector invention showing a Radial Cantilevered Flange 145 closely adjacent and joining a Compound Cantilever Flange 150 attached to a Body 160. Also shown is a Radial Cantilevered Flange 165 attached to Body 160. Not shown, but similar to the connector shown in FIG. 9, a second compound cantilevered flange located adjacent the Radial Cantilevered Flange 165. In this embodiment, Wings 170 are attached to Body 160 to facilitate insertion into a mating tapered shaft connector.

1) A tapered bore connector comprising:
a) a body having a conical bore that forms an opening of predetermined diameter in said body; said conical bore has a central axis; and
b) at least one compound cantilevered flange attached to said body;
i) said compound cantilevered flange extends a predetermined distance from said body in an outward radial direction relative to said axis of said conical bore; and
ii) said compound cantilevered flange extends a predetermined distance from said body in a circumferential direction relative to said axis of said conical bore;
whereby the compound cantilevered flange is deflected when the tapered bore connector is fully engaged with a compatible tapered shaft connector having an internally threaded locking collar thereby generating an additional restraining force that dissuades premature disconnection.

2) The tapered bore connector of claim 1 further including at least one radial cantilevered flange attached to said body; said radial cantilevered flange extends in an outward radial direction relative to said axis of said conical bore;
whereby said radial cantilevered flange limits the deflection of said compound cantilevered flange when said tapered bore connector is fully engaged with a compatible tapered shaft connector having an internally threaded locking collar.

3) The tapered bore connector of claim 2 wherein the said radial cantilevered flange is closely adjacent said compound cantilevered flange.

4) The tapered bore connector of claim 2 wherein
a) the said radial cantilevered flange has a first bottom edge having a first predetermined helix angle and
b) said compound cantilevered flange has a second bottom edge having a second predetermined helix angle and
c) said first predetermined helix angle is equal to or greater than said second predetermined helix angle.
whereby when said tapered bore connector is engaging with a compatible tapered shaft connector having an internally threaded locking collar, the compound cantilevered flange is first deflected and the radial cantilevered flange is second deflected to limit the deflection of the compound cantilevered flange.

5) The tapered bore connector of claim 4 where the first helix angle is about twenty five to thirty degrees.

6) The tapered bore connector of claim 4 where the second helix angle is about zero to five degrees.

7) The tapered bore connector of claim 1 wherein exactly two compound cantilevered flanges are attached to said body and spaced approximately 180 degrees apart relative to said axis of said conical bore.

8) The tapered bore connector of claim 1 wherein the conical bore of said body has an included angle between five degrees and seven degrees.

9) The tapered bore connector of claim 1 wherein the connector is composed of a thermoplastic resin.
